(19) United States
(12) Patent Application Publication
(10) Pub. No.: US 2007/0179438 A1
(43) Pub. Date: Aug. 2, 2007

(54) Surge suppression method

(75) Inventor: Michael D. Morgan, Costa Mesa, CA (US)

Correspondence Address: ALCON, IP LEGAL, TB4-8, 6201 SOUTH FREEWAY, FORT WORTH, TX, 76134, US

(73) Assignee: Alcon, Inc.

(21) Appl. No.: 11/342,743

(22) Filed: Jan. 30, 2006

(51) Int. Cl. A61M 31/00; A61M 37/00

(52) U.S. Cl. 604/67; 604/131

(57) ABSTRACT

A method of variably restricting flow through an aspiration line so as to reduce post occlusion break surge. The method involves attaching a portion of the aspiration line at the proximal end of the handpiece to a device that can variable twist the aspiration line. Such twisting reducing the interior diameter of the aspiration line, thereby restricting flow through the line.

### BACKGROUND OF THE INVENTION

This invention relates generally to the field of phacoemulsification and more particularly to aspiration systems used during phacoemulsification.

The human eye in its simplest terms functions to provide vision by transmitting light through a clear outer portion called the cornea, and focusing the image by way of the lens onto the retina. The quality of the focused image depends on many factors including the size and shape of the eye, and the transparency of the cornea and lens.

When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an IOL.

In the United States, the majority of cataractous lenses are removed by a surgical technique called phacoemulsification. During this procedure, a thin phacoemulsification cutting tip is inserted into the diseased lens and vibrated ultrasonically. The vibrating cutting tip liquefies or emulsifies the lens so that the lens may be aspirated out of the eye. The diseased lens, once removed, is replaced by an artificial lens.

A typical ultrasonic surgical device suitable for ophthalmic procedures consists of an ultrasonically driven handpiece, an attached cutting tip, and irrigating sleeve and an electronic control console. The handpiece assembly is attached to the control console by an electric cable and flexible tubings. Through the electric cable, the console varies the power level transmitted by the handpiece to the attached cutting tip and the flexible tubings supply irrigation fluid to and draw aspiration fluid from the eye through the handpiece assembly.

The operative part of the handpiece is a centrally located, hollow resonating bar or horn directly attached to a set of piezoelectric crystals. The crystals supply the required ultrasonic vibration needed to drive both the horn and the attached cutting tip during phacoemulsification and are controlled by the console. The crystal/horn assembly is suspended within the hollow body or shell of the handpiece by flexible mountings. The handpiece body terminates in a reduced diameter portion or nosecone at the body's distal end. The nosecone is externally threaded to accept the irrigation sleeve. Likewise, the horn bore is internally threaded at its distal end to receive the external threads of the cutting tip. The irrigation sleeve also has an internally threaded bore that is screwed onto the external threads of the nosecone. The cutting tip is adjusted so that the tip projects only a predetermined amount past the open end of the irrigating sleeve. Ultrasonic handpieces and cutting tips are more fully described in U.S. Pat. Nos. 3,589,363; 4,223,676; 4, 246, 902; 4,493,694; 4,515,583; 4,589,415; 4,609,368; 4,869,715; 4,922,902; 4,989,583; 5,154,694 and 5,359,996, the entire contents of which are incorporated herein by reference.

In use, the ends of the cutting tip and irrigating sleeve are inserted into a small incision of predetermined width in the cornea, sclera, or other location. The cutting tip is ultrasonically vibrated along its longitudinal axis within the irrigating sleeve by the crystal-driven ultrasonic horn, thereby emulsifying the selected tissue in situ. The hollow bore of the cutting tip communicates with the bore in the horn that in turn communicates with the aspiration line from the handpiece to the console. A reduced pressure or vacuum source in the console draws or aspirates the emulsified tissue from the eye through the open end of the cutting tip, the cutting tip and horn bores and the aspiration line and into a collection device. The aspiration of emulsified tissue is aided by a saline flushing solution or irrigant that is injected into the surgical site through the small annular gap between the inside surface of the irrigating sleeve and the cutting tip.

One possible complication associated with cataract surgery is anterior chamber collapse following an occlusion break. Occlusion of the phacoemulsification tip can occur when a piece of lens material fully covers the distal aspiration port. When an occlusion occurs, vacuum can build in the system aspiration line so that when the occlusion eventually breaks, a sudden surge occurs, drawing fluid and lens material out of the eye and into tip aspiration port. When fluid is draw out of the eye faster than it can be replaced, the eye can soften and collapse.

One way to reduce surge after an occlusion break is to reduce the internal diameter of the aspiration line. Reducing the internal diameter of the aspiration line can affect the fluidic performance of the system and makes the aspiration line susceptible to plugging and clogging. To reducing the chances of clogging, some have suggested adding a device to grind up the lens material being aspirated (see, for example PCT Publication No. WO 02/19896 A2). Such devices increase the cost and complexity of the system. Others have suggested a screen or filter to filter out the larger particles (see, for example, U.S. Patent Publication No. 2002/0128560). Still others have suggested that instead of reducing the diameter of the aspiration line, that the routing of the aspirant be circuitous (see for example, U.S. Patent Publication Nos. US 2003/0236508 A1 and US 2004/0064085 and U.S. Pat. Nos. 6,478,781 and 6,719,011 B2). A filter or circuitous routing of the aspirant flow can themselves cause plugging and sterility issues.

Therefore, a need continues to exist for a simple and reliable aspiration system that reduces fluid flow surges.

### BRIEF SUMMARY OF THE INVENTION

The present invention improves upon the prior art by providing a method of variably restricting flow through an aspiration line so as to reduce post occlusion break surge. The method involves attaching a portion of the aspiration line at the proximal end of the handpiece to a device that can variable twist the aspiration line. Such twisting reducing the interior diameter of the aspiration line, thereby restricting flow through the line.

Accordingly, one objective of the present invention is to provide a method of variably restricting flow through an aspiration line.

Another objective of the present invention is to provide a method to reduce post occlusion break surge.

These and other advantages and objectives of the present invention will become apparent from the detailed description and claims that follow.

### BRIEF DESCRIPTION OF THE DRAWINGS

FIG. 1 is a perspective view of a handpiece and control console that may be used with the present invention.

FIG. 2 is an enlarged perspective view of a phacoemulsification handpiece modified to practice the method of the present invention.

FIG. 3 is a cross-sectional view of one embodiment of an aspiration line that may be used with the method of the present invention.

### DETAILED DESCRIPTION OF THE INVENTION

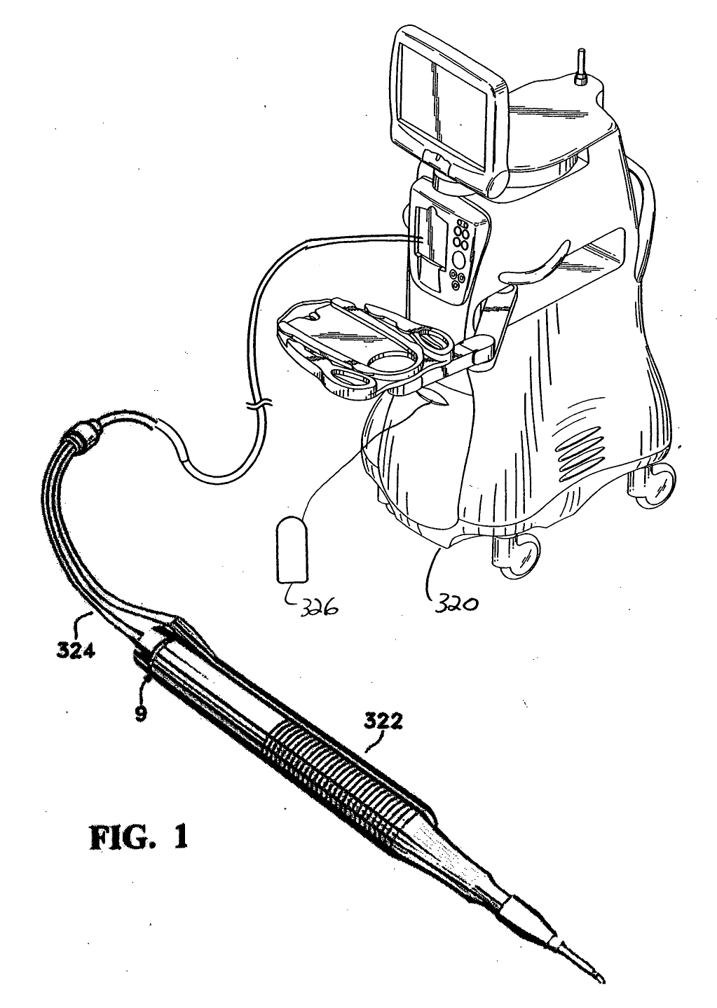

As best seen in FIG. 1, surgical console 320 suitable for use with the present invention may be any commercially available surgical control console such as the INFINITI® surgical systems available from Alcon Laboratories, Inc., Fort Worth, Tex. Console 320 is connected to handpiece 9 through irrigation line 322 and aspiration line 324, and the flow through lines 322 and 324 is controlled by the user, for example, via footswitch 326. Aspiration line generally is constructed from a flexible material such as silicone rubber.

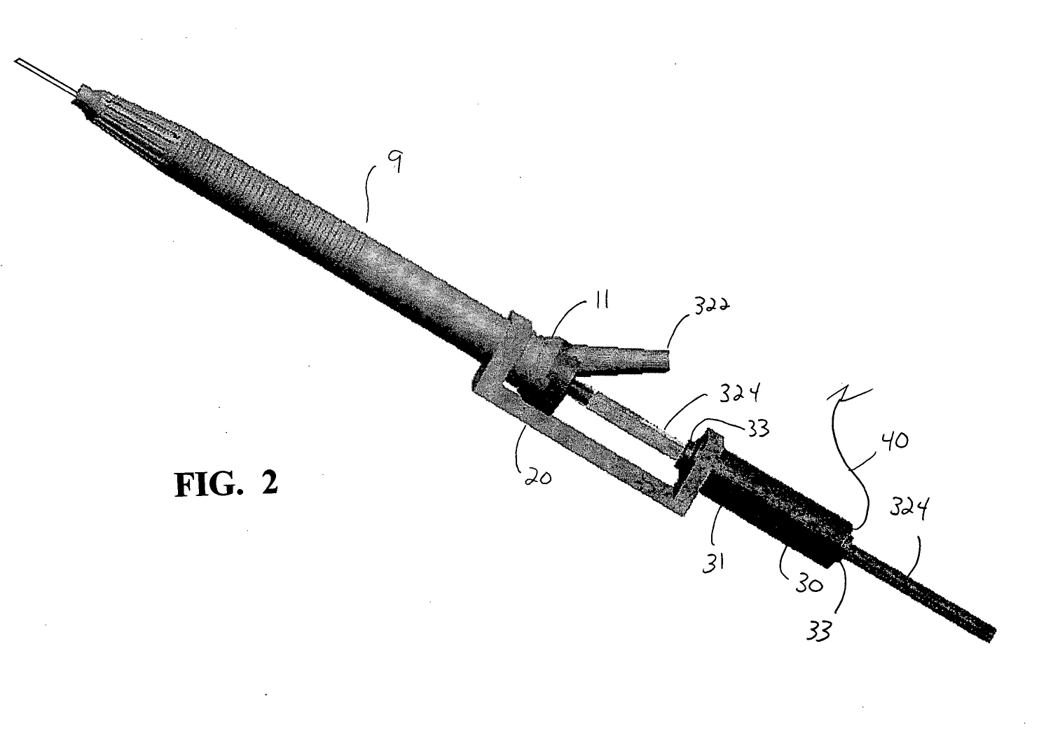

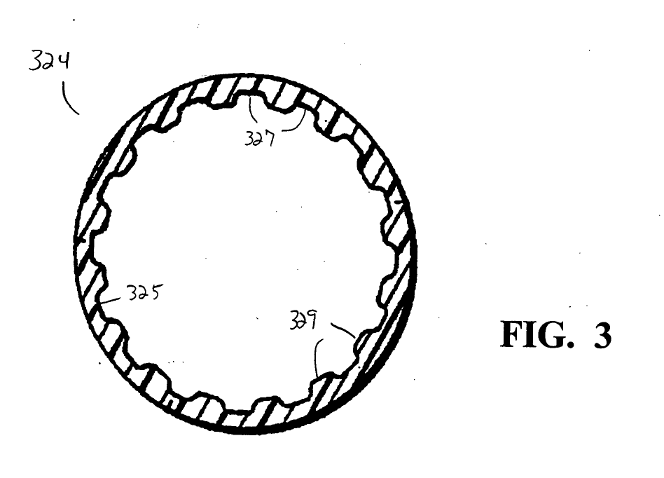

As best seen in FIG. 2, handpiece 9 has been modified to practice the method of the present invention. Attached to proximal end 11 of handpiece 9 via connector 20 is device 30. Aspiration line 324 exits proximal end 11 of handpiece 9 and is inserted through device 30. Aspiration line 324 is rigidly held with respect to inner barrel 33 of device 30 by friction or some other suitable attachment method. Connector 20 maintains outer shell 31 of device 30 rigidly with respect to handpiece 9, while allowing inner barrel 33 of device 30 to rotate relative to handpiece 9 via an electric motor or other suitable drive mechanism (not shown). Device 30 may communicate with and receive power from console 320 via electrical cable 40. One skilled in the art will recognize that device 30 may be external to handpiece 9, as shown in FIG. 2 or mounted internal to handpiece 9 or on console 320 (not shown). As best seen in FIG. 3, inner wall 325 of aspiration line 324 may contain longitudinal grooves 327 or ridges 329 that assist in providing more consistent reduced average flow when aspiration line 324 is twisted.

In use, console 320 monitors the vacuum level in an aspiration line 324 in a manner well-known in the art. When console 320 senses a vacuum level in aspiration line 324 consistent with an occlusion event, or the onset of an occlusion event, a signal is sent to device 30 via cable 40 causing inner barrel 33 to rotate. Such rotation of inner barrel 33 causes twisting of aspiration line 324 between handpiece 9 and device 30, thereby reducing the internal diameter of aspiration line 324 and restricting aspiration flow through aspiration line 324. When an occlusion event is no longer of concern, inner barrel 33 may be caused to counter-rotate, thereby untwisting aspiration line 324 to whatever degree desired and allowing aspiration flow through aspiration line 324 to return to more normal flow. If desired, aspiration line 324 can be twisted and untwisted in a serial or pulsed manner so as to produce a more consistent reduced flow condition.

This description is given for purposes of illustration and explanation. It will be apparent to those skilled in the relevant art that changes and modifications may be made to the invention described above without departing from its scope or spirit.

I claim:

1. A method of varying the flow through an aspiration line, comprising the steps of:
a) providing a handpiece, the handpiece having an aspiration line, at least a portion of the aspiration line being flexible;
b) monitoring a vacuum level in an aspiration line;
c) varying a flow through the aspiration line in response to the monitored vacuum level by twisting at least the flexible portion of the aspiration line.

2. The method of claim 1 wherein the flow through the aspiration line is varied in response to a sensed occlusion event at the handpiece.

3. The method of claim 1 wherein the twisting of at least the flexible portion of the aspiration line occurs external to the handpiece.

4. The method of claim 1 wherein the inner wall of the flexible portion of the aspiration line has grooves and/or ridges to provide more consistent reduced flow when the tubing is twisted.

5. The method of claim 1 wherein the twisting is performed in a pulsed manner in order to produce a consistent reduced average flow condition.
